(19) United States
(12) Patent Application Publication
(10) Pub. No.: US 2014/0275763 A1
(43) Pub. Date: Sep. 18, 2014

(54) PARTIALLY DISPOSABLE ENDOSCOPIC DEVICE

(71) Applicant: LUCENT MEDICAL SYSTEMS, INC., Kirkland, WA (US)

(72) Inventors: Curtis S. King, Kirkland, WA (US); Steve Vincent, Kirkland, WA (US); Robert N. Golden, Kirkland, WA (US); Fred E. Silverstein, Seattle, WA (US)

(73) Assignee: Lucent Medical Systems, Inc., Kirland, WA (US)

(21) Appl. No.: 13/839,198

(22) Filed: Mar. 15, 2013

(51) Int. Cl. A61B 1/00; A61B 1/015; A61B 1/06; A61B 1/005; A61B 1/05

(52) U.S. Cl. CPC A61B 1/00105; A61B 1/0052; A61B 1/00103; A61B 1/00144; A61B 1/05; A61B 1/0676; A61B 1/00016; A61B 1/015; A61B 1/0057
USPC 600/103; 600/147; 600/110; 600/146; 600/149

(57) ABSTRACT

An endoscopic device having a one-handed, either-handed steering mechanism is presented. The endoscopic device includes some components that are sterile and used on only a single patient. The single-use components are medically disposed of after exposure to a non-sterile environment such as an internal body cavity of a patient. The endoscopic device also includes some reusable components that are not exposed to a non-sterile environment.

### BACKGROUND

1. Technical Field

The present disclosure generally relates to an endoscopic-type instrument having disposable components and reusable components and methods to use the instrument. More particularly, but not exclusively, the present disclosure relates to a symmetric endoscopic-type instrument wherein the disposable components can be controlled with either a right hand or a left hand.

2. Description of the Related Art

In many medical procedures, a medical practitioner accesses an internal cavity of a patient. In some cases, the medical practitioner accesses the internal cavity for diagnostic purposes. In other cases, the practitioner accesses the cavity to provide treatment. In still other cases, different therapy is provided.

In one common procedure, a medical practitioner places a medical device (e.g., a medical tube) into the body of a patient. The medical device is conventionally passed into the body through the patient's mouth or nasal cavity, but the device can also be passed through a different orifice or another surgically made entry point (e.g., by incision or puncture).

The success of the medical procedure often depends on the proper placement of the medical device. In many cases, an endoscope can help to improve the chance for successful placement of the medical device. For example, in a percutaneous endoscopic gastrostomy (PEG) medical procedure, an endoscope is used to assist in the placement of the medical device (i.e., a medical tube) through the abdominal wall of the patient. When the medical device is a medical tube placed with a PEG procedure, the medical device may be broadly described as a “PEG tube.” One such PEG tube is a feeding tube, and the medical practitioner uses a PEG procedure to place the feeding tube (i.e., the PEG tube) into the stomach of a patient who cannot swallow liquids or solids. In some instances, a PEG tube is used to facilitate the placement of a feeding tube into the small bowel.

FIG. 1A illustrates a human patient 10. The nasal cavity 12 and throat 14 provide access pathways to the esophagus 16, which provides a pathway to an entry point (a diaphragm, not shown) in the stomach 18. The pylorus 20 provides an exit point from the stomach and an entry point into the small bowel 22. In FIG. 1A, a PEG tube 24 is illustrated as placed in the patient's stomach 18.

FIG. 1B illustrates in more detail the placement of the PEG tube 24 (FIG. 1A) in the patient's stomach 18. The PEG tube 24 passes through the skin 26, a fat layer 28, muscle 30, and the stomach wall 32. Inside the stomach 18, when the PEG tube 24 is placed, a mushroom catheter tip 34 of the PEG tube 24 provides an entry point into the stomach 18 for nutrients, medicine, or other therapeutic agents. The mushroom catheter tip 34 also provides a flange to which the body of the PEG tube 24 applies backwards pressure to an internal bumper 36. The internal bumper 36 forms a tight seal to prevent any of the stomach's contents from escaping out through the stomach wall 32 and to further prevent foreign materials from entering the stomach 18 through the stomach wall 32. A corresponding external bumper 38 includes a flange to which the body of the PEG tube 24 applies forward pressure thereby forming a seal at the surface of the skin 26. A clamp and adapter 40 controls the flow of liquids into and out of the patient's body.

FIG. 2 illustrates steps of a PEG procedure to place a PEG tube 24 into a patient's stomach 18. Traditionally, a PEG tube 24 can be placed using endoscopic guidance. An endoscope 42 is used in the PEG procedure.

The endoscope 42 includes a flexible tube 44 having one or more pathways axially formed within the flexible tube 44. The pathways include one endpoint on the proximal end 46 of the flexible tube 42 and a second endpoint formed on the distal end 48 of the flexible tube 42. One of the pathways includes a fiber-optic or other image passing cable with a lens or lens system placed at the distal end 48 of the flexible tube 44. In the base of the endoscope 42 or coupled thereto, a camera captures still pictures or moving video of that which is in front of the lens at the distal end 48 of the flexible tube 44. Other pathways in the endoscope 42 may be used to pass water, air, suction, or certain medical tools. The base of the endoscope 42 conventionally includes controls to steer the distal end 48 of the endoscope 42 orto control the other functions of the endoscope 42.

When the PEG procedure to place a PEG tube 24 begins, the distal end 48 of the endoscope's flexible tube 44 is passed through the patient's mouth and throat and into the esophagus 16. Using the camera tools, the medical practitioner is able to observe that the patient's esophagus 16 is without obstruction, diverticula, or other medical concerns. The medical practitioner advances the distal end 48 into the patient's stomach 18. The medical practitioner can use the tools of the endoscope 42 to inspect the stomach 18 and locate a suitable area 50 for the gastrostomy. During the inspection, the stomach 18 may be inflated by passing air through a lumen in the flexible tube 44, which allows the medical practitioner to see that the selected area 50 can be distended and that a PEG tube 24 placed in the selected area 50 will avoid interference with the pylorus 20. In this manner, a medical practitioner uses an endoscope 42 to select an area 50 of the lower body of the stomach or antrum (the gastric wall) that is particularly suitable for the PEG tube 24 placement.

In another step of the traditional PEG tube 24 placement procedure, the medical practitioner shines light 52 out from endoscope 42. In a darkened room, the light 52 can be seen through the patient's skin by a second medical practitioner (who may be any person trained in such medical procedures). Based on where the light is seen, the second medical practitioner can determine that the selected area 50 is in a reasonable location of the patient's body, e.g., not above the ribs. If the second medical practitioner is unable to see the light 50, the first medical practitioner can move the distal end 48 of the endoscope 42. By advancing or withdrawing the flexible tube 44 while also manipulating the steering controls, the medical practitioner can orient the distal end 48 of the flexible tube 44 of the endoscope 42 in three dimensions. If necessary, the medical practitioner can use the endoscope's tools to direct a stream of water over the lens and a lighting element in the distal end 48. Having thereby “cleaned” the lens and light source, the medical practitioner can use the tools to select a more preferable area 50 for the gastrostomy.

In addition or alternatively, the second medical practitioner may push a finger or another object into the skin of the patient 10 in the area of the stomach 18. The first medical practitioner can watch the images produced by the endoscope's camera to see an indentation in the stomach wall where the second medical practitioner is pressing. In this way, the medical practitioners can also select an area 50 for the gastrostomy.

Upon selection of a preferable area 50, and confirmation from inside the stomach 18 and outside the patient 10 that the preferred area 50 is suitable, the second medical practitioner will then make a small incision in the skin of the patient 10. A needle is inserted into the patient 10 at the site of the incision, and the needle is advanced through the fat layer 28, muscle 30, and stomach wall 32 to penetrate the area 50 selected by the practitioners. Using the camera tools of the endoscope 42, the medical practitioner expects to see the needle enter the stomach 18 in the selected area 50.

In a subsequent step in the traditional placement of a PEG tube 24, a medical wire can be passed through a lumen in the needle. The medical practitioner will use a snare tool in the endoscope to grasp the wire firmly. The endoscope and snare are then withdrawn from the patient's mouth, thereby pulling the wire from the outside of the patient's abdomen, through the needle into the stomach 18, up through the esophagus 16, and out of the patient's mouth. The part of the wire that extends out from the patient's mouth is subsequently attached to the PEG tube 24.

In yet another step in the traditional placement of a PEG tube 24, once the wire is successfully passed through the patient 10, a PEG tube 24 is secured to the end of the wire extending from the patient's mouth. The PEG tube 24 is guided into the patient's mouth and pulled into the patient's stomach 18 as the wire is pulled from the end that passed through the needle. Once the PEG tube 24 is in the stomach 18, the tube is pulled partially through the gastric and abdominal walls until the internal bumper 36 of the PEG tube 24 is snug against the gastric mucosa of the stomach 18. The external bumper 38 is similarly pressed snug against the patient's skin and secured in place for example with a stitch.

Upon placement of the PEG tube 24, the flexible tube 44 of the endoscope 42 can be re-advanced into the stomach 18 of the patient 10 and used to verify effective placement of the PEG tube 24. In other traditional PEG tube placement procedures, endoscopy is not in the final step, or endoscopy may not be used at all. Instead, x-ray may be used to verify a proper placement of the PEG tube 24 or to select a particularly suitable location 50 in the patient's body (e.g., the stomach) for the introduction of the PEG tube 24.

FIG. 3 illustrates a conventional endoscope 42. The endoscope 42 includes a flexible tube 44 having a proximal end 46 and a distal end 48. The flexible tube 44 has a desired degree of torque stability and mechanical controllability. In order to provide this feature, the flexible tube 44 employs a multi-clad armor sheath having interlocking segments arranged in a helical-anti-helical fashion. In this arrangement, as one segment begins to twist, an opposing segment locks against it, thereby opposing the twist.

The endoscope 42 includes a handle portion 56 coupled to the flexible tube 44. The handle 46 of endoscope 42 illustrated in FIG. 3 is asymmetrical and configured for left-handed operation. In other cases, an endoscope is configured for right-handed operation. Generally speaking, the body of a conventional endoscope control handle is arranged asymmetrically such that any particular endoscope is configured for either right-handed operation or left-handed operation but not both.

In the endoscope 42 of FIG. 3, a medical practitioner will place the handle portion in the palm of his left hand. A first material port 58 will rest in the space between the thumb of the medical practitioner's left hand and his index finger. The medical practitioner's fingers will wrap around the handle 56 of the endoscope 42 in position to operate a visualization control 60 and a set of trumpet controls including a first control 62, a second control 64, and a third control 66. The medical practitioner's left thumb will wrap around the handle 56 in an opposite direction as his fingers such that the handle portion 56 of the endoscope 42 is in firm control of the medical practitioner's left hand. The left thumb of the medical practitioner is positioned to operate a first steering control wheel 68 and a second steering control wheel 70. Alternatively, when the medical practitioner is holding the endoscope 42 as so described, he can operate the steering control wheels 68, 70 with his right hand. The endoscope 42 of FIG. 3 further includes a second material port 72 and a tool port 74, both of which are arranged at a lower section of the endoscope's handle 56. A visualization port 76 is arranged at the top section of the endoscope's handle 56.

The distal end 48 of the flexible tube 44 includes a flexible tip 54. The flexible tip 54 may be controlled in three dimensions using the first steering control wheel 68 and the second steering control wheel 70. Extending from inside the handle 56 of the endoscope 42, steering cables are coupled to the first and second control wheels 68, 70. The steering cables extend into the proximal end 46 of the flexible tube 44. The steering cables pass under a jacket in the flexible tube 44 to the distal end 48 of the flexible tube 44 where they are fastened to the flexible tip 54. One of the two steering wheels, when rotated, is arranged to move the flexible tip 54 in a left and right direction. The other of the two steering wheels, when rotated, is arranged to move the flexible tip 54 in an up and down direction. Accordingly, when the medical practitioner uses his left thumb to rotate the first and second steering wheels 68, 70, the flexible tip 54 of the endoscope 42 can be controlled in three dimensions. Furthermore, as the medical practitioner advances and withdraws the flexible tube 44 within a patient's body, the flexible tip 54 of the endoscope 42 can be “aimed” in any direction along the flexible tube's path of travel. A tension control knob 78 can provide further control for the medical practitioner to lock, unlock, or change the amount of force necessary to rotate the steering wheels 68, 70.

The flexible tip of the endoscope 42 includes a distal end assembly 80. The distal end assembly 80 is illustrated in detail A. The distal end assembly 80 includes a tool port orifice 82. The tool port orifice 82 is a termination point of a lumen that passes through the flexible tube 44 to an entry point of a tool port 74 in the handle portion 56 of the endoscope 42. A medical tool, for example a biopsy collection device, can be passed by the medical practitioner through the tool port 74. From outside of the patient, the medical practitioner cannot advance the medical tool into the body of the patient. The medical tool advances out of the tool port orifice 82 where it can be used in a medical procedure. In another example, the medical practitioner can pass a medical tool snare device through the tool port 74 to grab a guide wire used in a PEG tube placement procedure.

The distal end assembly 80 also illustrates the termination points of several other lumens that passed through the flexible tube 44. With his left hand, the medical practitioner can control the operational features of the end assembly 80. For example, in some cases, a water source, an air source, and a suction source are coupled to the second material port 72. A light source is coupled to the first material port 62. Using the trumpet controls 62, 64, 66, the medical practitioner can pass water or air out of the water and air nozzle, and the medical practitioner can engage or disengage a suction source to collect material through the suction inlet 90. Using the visualization control 60, the medical practitioner can enable a light source that supplies light passed from the light source aperture 86. Also using the visualization control 60, the medical practitioner can focus, collect, or pass still or moving images via the visualization port 76. In some cases, the endoscope 42 includes optical visualization components. In such cases, the visualization port 76 is generally an eyepiece that the medical practitioner can look through, and the visualization control 60 performs mechanical focusing control. In other cases, the endoscope 42 includes electronic visualization components, and in these cases, the visualization control 60 directs an electronic camera system.

The endoscope 42 of FIG. 3 is representative of a conventional endoscope. In other conventional endoscopes, air, water, suction, and other therapeutic agents may be passed through different ports and controlled with different buttons than described herein. Additionally, the end assembly 80 may also be configured differently than what is illustrated. The endoscope 42 is not necessarily drawn to scale. Instead, the endoscope 42 of FIG. 3 is presented to illustrate particular structures and features common to many conventional endoscopes.

### BRIEF SUMMARY

In accordance with some embodiments described herein, an endoscopic device has an image sensor arranged in a functional module at the steerable tip portion of a flexible tube attached to the endoscopic device. The tip can be steered in a back and forth direction of a common plane. The steering mechanism is configured for one-handed, either-handed use. The flexible tube is torque stable. As the flexible tube is rotated and the tip portion is steered, the functional module can be aimed in any direction in three dimensions. The functional module may include various controllable nozzles, ports, and electronic features such as an image sensor and lights. The features of the functional module are directed by a control module located in the handle of the endoscopic device. The handle of the endoscopic device and the flexible tube are sterile and configured for use on a single patient. The handle of the endoscopic device and the flexible tube are disposed of after they are used in a single medical procedure. The control module that directs the operations of the functional module is reusable.

In a first embodiment, an endoscopic device includes a disposable control handle having a sealable recess therein, a disposable flexible tubular member coupled to the control handle, and a non-disposable electronics control module configured for removable placement within the sealable recess.

In a second embodiment, a method to use a partially disposable endoscopic device is disclosed. The method includes the act of arranging a sterile disposable control handle having a sealable recess therein in the vicinity of a patient's body, the sterile disposable control handle having a sterile disposable flexible tubular member coupled thereto. The method also includes the acts of opening the sealable recess in the disposable control handle and introducing a non-disposable electronics control module into the sealable recess. An act of performing a medical procedure that includes passing the sterile disposable flexible tubular member into the patient's body and controlling at least one feature of the disposable flexible tubular member with the disposable control handle is also included in the method. Acts of re-opening the sealable recess in the disposable control handle, removing the non-disposable electronics control module from sealable recess, and disposing of the sterile disposable control handle and the sterile disposable flexible tubular member are also included in the method.

In another embodiment, an endoscopic device includes a control handle configured for single-handed, either-handed operation, and a steering mechanism cooperatively assembled with the control handle, the steering mechanism arranged to steer at least one portion of a flexible tubular member attachable to the steering mechanism.

These features with other objects and advantages which will become subsequently apparent reside in the details of construction and operation as more fully described hereafter and claimed, reference being had to the accompanying drawings forming a part hereof.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. One or more embodiments are described hereinafter with reference to the accompanying drawings in which:

FIG. 1A illustrates a human patient wherein a PEG tube is illustrated as placed in the patient's stomach;

FIG. 1B illustrates in more detail the placement of the PEG tube of FIG. 1A in the patient's stomach;

FIG. 2 illustrates steps of a PEG procedure to place a PEG tube into a patient's stomach;

FIG. 3 illustrates a conventional endoscope;

FIG. 4 is a perspective view of one embodiment of a partially disposable endoscopic device;

FIGS. 5A-5C each illustrate three views of the steering mechanism of the partially disposable endoscopic device of FIG. 4;

FIG. 6 is an exploded view of the partially disposable endoscopic device of FIG. 4;

FIGS. 7A-7E show several views of the symmetrical steering mechanism cover and various other structures associated with the symmetrical steering mechanism;

FIG. 8 is an exploded view of a symmetrical steering mechanism embodiment from a rear perspective;

FIG. 9 shows one embodiment of a steering cable drive pulley means;

FIG. 10 shows another embodiment of a steering cable drive pulley means;

FIG. 11 shows an embodiment of a steering cable rack and pinion drive means;

FIGS. 12A and 12B each illustrate three views related to deflection of the steerable tip portion as directed by operation of the rack and pinion drive means of FIG. 11;

FIG. 13 is an overlay section view of a partially disposable endoscopic device handle portion;

FIG. 14 is a perspective view of an exemplary control module;

FIG. 15 is an exploded view of the control module of FIG. 14;

FIG. 16 illustrates a top view and a section view of the control module of FIG. 14;

FIG. 17 presents front and back views of the control module embodiment of FIG. 14; and

FIG. 18 illustrates a partially disposable endoscopic device embodiment in use in a medical procedure.

### DETAILED DESCRIPTION

Broadly speaking, an endoscope is an instrument used to see inside the body of a patient. A conventional medical endoscope includes a flexible tube, a functional control mechanism to direct the position of a distal end of the flexible tube, and a camera. The camera provides images of the internal body cavity to help the medical practitioner position the distal end of the flexible tube and confirm that the end is positioned at an acceptable location. The endoscope may also include other features as heretofore described.

Endoscopes are useful in the diagnosis and treatment of many medical conditions. Some medical procedures and therapies can only be performed with an endoscope or endoscopic type tool. As it turns out, however, endoscopes can be traced as one cause of a high number of healthcare provider associated diseases.

When the endoscope is used in a medical procedure, the flexible tube of an endoscope is generally directed into an internal body cavity of a patient. During its use in the procedure by a medical practitioner, the endoscope may acquire high levels of microbial contamination. The microbial contamination may include infectious agents or any number of harmful bacterial and viral microorganisms. In some cases, an endoscope used in a medical procedure on one patient is contaminated, improperly or insufficiently disinfected, and used in a medical procedure on another patient. In such cases, the health of the second patient is put at risk of microbial transmission or disease.

Medical practitioners work to prevent the spread of nosocomial infection and disease by following strict procedures to clean and disinfect an endoscope. Unfortunately, most conventional endoscopes (e.g., bronchoscopes, colonoscopes, gastrointestinal endoscopes, nasopharyngoscopes, sigmoidoscopes, and the like) are heat sensitive and cannot be sterilized. Instead, the endoscopes are cleaned with other procedures and wiped or even bathed in high-level disinfectants.

In spite of rigid attempts to effectively clean endoscopes, some patients suffer injury, illness, and even death as a result of an endoscope that carries pathogens from one patient to another. In 2010, the ECRI Institute cited endoscopic contamination as one of the top 10 health risks in a document entitled “Top 10 Health Technology Hazards for 2011,” Reprinted from Volume 39, Issue 11, November 2012 by the ECRI Institute (www.ecri.org). A seminal work that studied and described the problem of improperly cleaned endoscopes is “Transmission of Infection by Gastrointestinal Endoscopy and Bronchoscopy,” from the Annals of Internal Medicine, 1993; 118:117-128 by the American College of Physicians, authored in part by one of the present inventors.

FIG. 4 is a perspective view of one embodiment of a partially disposable endoscopic device 100. The partially disposable endoscopic device 100 embodiment includes a disposable control handle shell (i.e., “handle body,” “handle portion,” etc.) 102 and a flexible tubular member (i.e., “tube body,” “flexible tube,” etc.) 104 coupled or coupleable to the handle body. A steerable tip portion 106 is integrated at a distal end of the tube body 104. The steerable tip 106 is controlled via symmetrical steering mechanism 108 integrated with the handle body 102. The endoscopic device embodiment 100 also includes a functional module 110 arranged at the steerable tip portion 106. The functional module 110 may include an image sensor module, a light source, nozzles, ports, apertures, and other functional features, which are later described in more detail. The functional module 110 is coupled to corresponding controls and structures located in the handle body 102. The functional module 110 may also be disposable.

The symmetrical steering mechanism 108 is illustrated in FIG. 4 as including a textured end cap. The illustrated shape is that of a frustum formed from a prolate spheroid, but other shapes are possible. In the present embodiment, the frustum configures the endoscopic device 100 for one-handed, either-handed operation. That is, a medical practitioner can position the endoscopic device 100 in either his left palm or his right palm and comfortably operate the device. As illustrated in FIG. 4, when the steering mechanism 108 is rotated in a first direction, a first steering cable 116a is withdrawn into the body of the handle portion 102. When the first steering cable 116a is pulled in such a manner, the steerable tip portion 106 is deflected in a first direction 118a in a first plane. When the steering mechanism 108 is rotated in a second direction opposite to the first direction, the second steering cable 116b is withdrawn into the body of the handle portion 102. Pulling the second steering cable 116b in such a manner deflects the steerable tip portion 106 in a second, opposite direction 118b in the first plane.

With respect to the embodiment of FIG. 4, the handle body 102 and the flexible tube 104, along with the components integrated therein (e.g., steerable tip 106, steering mechanism 108, and functional module 110), are configured for use in a single patient. I.e., the assembly made up by the handle body 102 and the flexible tube 104 is configured to be disposable.

The flexible tube 104 may be configured in many different ways. Flexible tubes 104 that are compatible with the handle body 102 may be formed having many different lengths and many different degrees of flexibility. In some cases, color coding or numerical marking is provided to help medical practitioners distinguish flexible tubes 104 of different lengths and flexibilities.

A flexible tube 104 generally exhibits properties of torque stability. The properties may be a function of the materials used to form the flexible tube 104. The properties may also be a function of structures embedded in the walls of the flexible tube 104. The torque stable nature of a flexible tube 104 permits a medical practitioner to rotate a proximal end of the flexible tube 104 with confidence that the distal end of the flexible tube 104 will rotate in a corresponding direction and to a corresponding degree. By way of example, a 24″ section of common copper plumbing pipe (standard K, nominal ½″ diameter, 0.625″ O.D., 0.049″ wall thickness) is considered torque stable, and a 24″ section of common rubber hose having the same dimensions is considered not torque stable.

Flexible tubes 104 may be formed of many different widths. In some cases, a control handle 102 will be arranged to receive a flexible tube having a single width throughout its length. Accordingly, as the width of a flexible tube increases, the entrance hole in the control handle 102 will also increase. In other embodiments, a flexible tube 104 may be formed with multiple widths. A first width at the proximal end of the flexible tube 104 will be formed to a standard size for compatibility with control handles 102 having an entrance hole of the standard size. At some point along the length of the tubular body 104, the width of the flexible tube 104 will change. The width may increase in some cases. The width may decrease in other cases. A small diameter flexible tube 104 may be suitable for a smaller patient and for passage in a smaller space, for example, an artery or a vein. A large diameter flexible tube 104 may be suitable for a larger patient and for passage in a larger space. Additionally, a larger diameter flexible tube 104 may accommodate more lumens and thereby more tools or lumens of a larger size and thereby larger tools.

In some cases, the steerable tip portion 106 of a flexible tube 104 may have a functional module 110 at the distal tip of the steerable tip portion 106. One non-limiting embodiment of a functional module 110 is illustrated in FIG. 4. In the embodiment, the functional module 110 includes a tool port orifice 192, a water nozzle 194, an air nozzle 196, an image sensor 198, a suction inlet 200, and a light source 202.

The functional module 110 may be integrated in the steerable tip portion 106 of the flexible tube 104 of the endoscopic device 100. The functional module 110 itself may be flexible or rigid. In some embodiments, the steerable tip portion has flexible baffles to define a bending profile for the tip. In other embodiments, the flexible baffles are a sheath covering a bending mechanism (e.g., a spring).

The functional module 110 may include an imaging module 198 and a light source 202. The imaging module 198 can be a charge couple device (CCD) video camera integrated circuit (IC) or some other type of image sensor. The light source 202 may include one or more light emitting diodes (LEDs), which can shine light through an optional window. The imaging/illumination modules 198, 202 respectively will also include an electrical interface assembly to provide power and control signals to the light source 202 and image sensor 198. A control module 112 may include optional electronic camera features. The light source 202 in the endoscopic device 100 permits the medical practitioner to see inside the cavity where the endoscopic device 100 has been placed, and the image sensor 198, if included, can capture and pass individual picture images or a video stream of the area in front of the flexible tip assembly 106.

The steerable tip portion 106 and thereby the functional module 110 can be moved in a plurality of directions when the steering mechanism 108 is manipulated and the control handle 102 is rotated, advanced, or withdrawn.

As illustrated in FIG. 4, the handle body 102 is arranged to receive a non-disposable (i.e., reusable) control module 112. The non-disposable control module 112 will typically include structures that support the features provided by the functional module 110. For example, the non-disposable control module 112 will include structures such as a battery (e.g., a rechargeable battery), a microprocessor, memory, communications port(s), a user interface, a wireless radio transceiver, electronic switches, a vibrator, a sound producing device, an electro-mechanical interface corresponding to one or more electro-mechanical interfaces in the handle body 102, and other control circuits and structures.

The handle body 102 of the endoscopic device 100 is arranged to receive the non-disposable control module 112 through an opening in the handle body 102. The opening in the handle body 102 is illustrated as being opposite the tube body 104 in FIG. 4, but other arrangements are possible. A hinged end cap 114 includes an interlock mechanism configured to mate with a corresponding interlock mechanism of the handle body 102. When the end cap 114 is opened, the non-disposable control module 112 can be inserted and removed from the handle body 102.

The endoscopic device 100 illustrated in FIG. 4 is partially disposable and symmetrically steerable. The particular features of the endoscopic device 100 are described in further detail in subsequent paragraphs.

FIGS. 5A-5C each illustrate three views of the steering mechanism of the partially disposable endoscopic device 100 of FIG. 4. In FIG. 5A, the steering mechanism 108 is illustrated in a view from the front. The steering mechanism 108 is biased to a deflection of 0 degrees as illustrated by the vertical arrow reference mark directed to the reference scale indicator of 0°. The reference indicators illustrated in FIG. 5A further illustrate a counterclockwise deflection to −90° and a clockwise deflection to 90°.

In some embodiments, a scale (e.g., a graduated scale) provides an indication of rotation of the steering mechanism 108. The graduated scale may have linear marks that provide a relative indication of motion. Alternatively, the scale may have actual measurement information that indicates how far the steerable tip portion 106 has moved. The scale may reflect millimeters, inches, fractions of inches, or some other unit of measure. In some cases, a vernier scale is also provided to more accurately indicate deflection. In yet other embodiments, no scales, arrows, needles, or reference marks of any kind are provided.

As illustrated in FIG. 5A, the steering mechanism 108 is arranged for rightward (clockwise) rotation and leftward (counterclockwise) rotation. The illustrated steering mechanism 108 includes shaped and textured tactile features. The illustrated shaped and textured features are not limiting and other shapes and in addition or alternatively other tactile features may also be configured in the external surface of the steering mechanism 108.

In operation, the steering mechanism 108 is configured for one-handed, either-handed operation. That is, the steering mechanism 108 is symmetrically arranged in the endoscopic device 100 for use by either a right hand or a left hand. For example, when a medical practitioner grasps the handle body 102 of the endoscopic device 100 in his left hand, the medical practitioner can use his left thumb or left thumb and left index finger to rotate the steering mechanism 108. Alternatively, if the medical practitioner grasps the endoscopic device 100 in his right hand, he can use his right thumb or right thumb and right index finger to rotate the steering mechanism 108.

The steering mechanism 108 is not limited to one-handed operation. In still another alternative, the medical practitioner can grasp the handle body 102 of the endoscopic device 100 with one hand and operate the steering mechanism 108 with his other hand. Other orientations can also be used by the medical practitioner to manipulate the steering mechanism 108.

FIG. 5A also illustrates a section view of a portion of the flexible tubular member 104. Steering cables 116a and 116b are illustrated. Consistent with the illustration of the steering mechanism 108 biased to a deflection of 0°, the steering cables 116a, 116b are illustrated as having no change in deflection (ΔL=0). Further consistent with the illustration of the steering mechanism 108 biased to a deflection of 0°, the steerable tip portion 106 is illustrated in a “straight” orientation.

In FIG. 5B, the steering mechanism 108 is rotated rightward to a deflection of about 60-70 degrees as illustrated by the arrow reference mark directed between the reference indicators of 0° and +90°. In the section view of FIG. 5B, a portion of the flexible tubular member 104 is illustrated in which steering cable 116a appears “shorter” than steering cable 116b by some length ΔL. The respective cable length difference coincides with the clockwise rotation of steering mechanism 108. Turning the steering mechanism 108 as indicated in FIG. 5B causes a rightward deflection of the steerable tip portion 106. FIG. 5B illustrates the steerable tip portion 106 deflected in a first direction 118a in a first plane.

In FIG. 5C, the steering mechanism 108 is rotated leftward to a deflection of about minus 60 to minus 70 degrees as illustrated by the arrow reference mark directed between the reference indicators of 0° and −90°. In the section view of FIG. 5C, a portion of the flexible tubular member 104 is illustrated in which steering cable 116a appears “longer” than steering cable 116b by some length ΔL. The respective cable length difference coincides with the counter-clockwise rotation of steering mechanism 108. Turning the steering mechanism 108 as indicated in FIG. 5C causes a leftward deflection of the steerable tip portion 106. FIG. 5C illustrates the steerable tip portion 106 deflected in a second direction 118b in the first plane.

As illustrated in FIGS. 5A-5C, a medical practitioner can rotate the steering mechanism 108 to cause deflection of the steerable tip portion 106 in a first plane. In some cases, the rotation of steering mechanism 108 has a one-to-one (1:1) corresponding relationship with the deflection of steerable tip portion 106. For example, if the steering mechanism 108 rotates to minus 45 degrees, the steerable tip deflects left to a corresponding minus 45 degrees; when the steering mechanism 108 rotates to plus 25 degrees, the steerable tip deflects left to a corresponding plus 25 degrees, and so forth.

In other cases, the rotation of steering mechanism 108 has a different relationship with the deflection of the steerable tip portion 106. In endoscopic devices where substantial accuracy in the deflection of the steerable tip portion 106 is desired, a greater rotation of the steering mechanism 108 will be necessary to deflect the steerable tip 106. In endoscopic devices where easy, one-handed operation may be desired, a smaller rotation of the steering mechanism 108 will cause a greater deflection of the steerable tip 106. In one embodiment, a one-to-one-point-five (1:1.5) relationship exists such that a 30 degree rotation of steering mechanism 108 causes a corresponding, proportional 45 degree deflection of the steerable tip 106. Other linear and even non-linear relationships between the steering mechanism 104 and the steerable tip 106 can also be provided.

FIG. 6 is an exploded view of the partially disposable endoscopic device 100 of FIG. 4. A disposable control handle shell (i.e., handle body) 102 and a symmetrical steering mechanism 108 are shown along with a non-limiting arrangement of internal structures to be described forthwith. A portion of the flexible tubular member (i.e., tube body) 104 is also shown with first and second steering cable sections 116a, 116b.

The outer shell of the disposable handle body 102 includes a handle body upper housing 120 and handle body lower housing 122. In FIG. 6, the non-disposable control module 112 is arranged to fit in a recess of the handle body lower housing 122, behind a hinged end cap 114, but such placement is illustrative only, and other arrangements are possible.

The symmetrical steering mechanism 108 is distinguishable from and integrated with the handle body 102 structures and tube body 104 structures in the embodiment shown in FIG. 6. Other arrangements are also possible. The embodiment of FIG. 6 is discussed for simplicity.

The symmetrical steering mechanism 108 is shown having a textured end cap (or cover) 124. The steering mechanism cover 124 is configured with a circular aperture (i.e., a hole) formed around a central axis of the frustum. The aperture may be circular, notched, toothed, hexagonal, or of some other shape. The aperture in the steering mechanism cover is arranged to receive a tube body outer guide flange 126, which cooperatively couples with a tube body inner guide flange 128.

In the endoscopic device 100 embodiment of FIG. 6, the tube body's outer and inner guide flanges 126, 128 are illustrated as permanently integrated with the tube body 104, but other arrangements are possible. For example, in other embodiments, the tube body's outer and inner guide flanges 126, 128 may be detachably configured to receive a tube body 104. That is, in some cases, a disposable handle body 102 may be separately manufactured and distributed from cooperating tube body 104 components. Differently sized and configured tube bodies 104 can be used with a common handle body 102 as the needs of a particular medical procedure or patient call for.

Along the same lines, in some cases, a tube body 104 and steering mechanism 108 are manufactured and distributed separately from another structure that includes the upper and lower handle body housings 120, 122 and internal structures such as the non-disposable control module 112. In these types of embodiments, a medical practitioner or other person can combine a preassembled tube body 104 and steering mechanism 108 having certain desirable properties with a handle shell 120, 122 and non-disposable control module 112 having standardized properties.

The tube body inner guide flange 128 also includes an integrated steering cable guide to direct a desirable coupling of first and second steering cables 116a, 116b into a steering cable drive pulley mounting chassis 130, which is configured to receive a pinion shaft/pinion gear structure 132.

The pinion gear and shaft 132 structure is arranged with an optional encoder 152 (e.g., a rotary encoder). When the encoder 152 is included, a position of the pinion gear shaft can be tracked to a known degree of precision. The tracking information may be electronically supplied to a control module for visual presentation to a medical practitioner, for electronic storage such that specific details of a medical procedure can be studied after the procedure is complete, or for other purposes.

When the partially disposable endoscopic device 100 is assembled, a ring gear 138 (FIG. 7E) located within the symmetrical steering mechanism cover 124 engages with pinion gear 132. Thus, upon assembly, when the steering mechanism cover 124 is rotated, pinion gear 132 will also differentially rotate. Pinion gear 132 includes an attached or integrated shaft, and as pinion gear 132 rotates, the attached shaft also rotates. The assembly of the steering mechanism 108 fixedly engages the pinion shaft 132 to a steering cable drive pulley 140 (FIG. 9) mounted in chassis 130. Steering cables 116a, 116b are engaged with the steering cable drive pulley mounted in chassis 130. Accordingly, when the steering mechanism cover 124 is rotated, one steering cable 116a or 116b is tensioned (while the other steering cable is released), and the steerable tip portion 106 of the partially disposable endoscopic device 100 is deflected in either a first direction or a second direction of a first plane.

In the embodiment of FIG. 6, an input/output (I/O) interface board 134 is coupled to the steering cable drive pulley mounting chassis 130. One or more user interface I/O structures 136 are arranged to pass electrical signals or tactile signals to the I/O interface board 134. For example, the user interface I/O structures 136 may include rubber “buttons” that pass physical key depressions made by a medical practitioner to an electro-mechanical switch mounted on the I/O interface board 134. Alternatively or in addition, user interface I/O structures may include LED light outputs, a clear window, a liquid crystal display (LCD), another type of display, a piezo or other audio device, a microphone, or any other type of I/O structure arranged to pass signals between the outside of the handle body 102 and the I/O interface board 134.

In some embodiments, the handle body housings 120, 122 and other structures of the partially disposable endoscopic device 100 are formed from a plastic material. In other embodiments, the structures are not formed of plastic. Generally speaking, the structures can be made from any of a wide variety of materials including moldable organic and inorganic materials that are pure or formed as a compound.

When the endoscopic device 100 is assembled, the internal components are hermetically sealed. The external housing features (e.g., upper housing 120, lower housing 122, front cover 124, hinged end cap 114) may optionally include rubber, silicon, or other sealing materials on their mating surfaces. Alternatively, the individual structures may have sufficiently acceptable tolerances such that a hermetic seal may be formed when the structures are assembled. In some cases, the assembled structures may be snap-fit with cooperative features on adjacent housings. In some cases, the structures are welded, glued, or otherwise permanently assembled. In still other cases, the housings may be screwed together or joined with some other distinct fastening mechanism.

The external housing features of the disposable control handle 102 (e.g., upper housing 120, lower housing 122, front cover 124, hinged end cap 114) are sterile before being used in a medical procedure. The disposable control handle 102 may be used only on a single patient. Accordingly, each patient that is exposed to a partially disposable endoscopic device 100 is assured that no contaminants from other patients are transferred.

The control handle features are typically sterilized at the time of manufacture. The sterilization procedure can be by heat, chemical, irradiation, or by some other means. In one embodiment, the sterilization procedure kills or otherwise eliminates all microbial matter present on the surface of the control handle features to a high sterility assurance level.

After sterilization, the sterile disposable control handle 102 can then be packaged in a manner that keeps the control handle free of pathogenic organisms until the control handle is to be used in a medical procedure.

In one method, a medical practitioner or other party prepares a partially disposable endoscopic device 100 for use in a medical procedure. Typically, the handler of sterile disposable control handle 102 has antiseptically washed and donned medically clean attire and sterile gloves. In an environment where the medical procedure is to take place, or optionally in a nearby antiseptic environment, the sterile disposable control handle 102 is removed from its packaging. Additionally, the non-disposable control module 112 is removed from its packaging. The control module 112 may be sterilized, disinfected, or sanitized. Subsequently, the access port of disposable control handle 102 (e.g., the hinged end cap 114) is opened to reveal a recess in the disposable control handle 102. As the control module 112 is inserted into the control handle 102, certain structural features of the control module 112 register with corresponding structural features in the recess of the control handle 102. In one embodiment, a set of spring-enabled electrical contacts (e.g., pogo pins) of one structure (e.g., the control module 112) engage into electro-mechanical contact with the other structure (e.g., the control handle 102). The handler of the endoscopic device 100 may then remove the present sterile gloves and replace them with new sterile gloves. The medical practitioner then performs the medical procedure.

Upon completion of the medical procedure, a handler of the partially disposable endoscopic device 100 may then prepare a sterile or other container to receive the control module 112. For example, the handler or some other person may open a plastic bag. Subsequently, the handler of the partially disposable endoscopic device 100 will access the recess in the control handle 102. For example, the handler depresses a locking tab of the control handle 102 or hinged end cap 114. The control module 112, via gravity, springed features, or other means, can be removed from the control handle 102 recess and deposited in the container arranged to receive it. In one example, the control module 112 is partially ejected via a spring-type mechanism when the hinged end cap 114 is open. In the example, the handler then “dumps” the control module 112 into a medically clean bag or other container, and the handler medically disposes of the control handle 102. The control module 112 is then sterilized, disinfected, or otherwise sanitized, upon which the control module can be used in another new, sterile control handle 102 for another patient's medical procedure. According to such a method, the partially disposable endoscopic device 100 includes a disposable control handle portion 102 and a reusable, non-disposable control module 112.

FIGS. 7A-7E show several views of the symmetrical steering mechanism cover 124 and various other structures associated with the symmetrical steering mechanism 108. In FIG. 7A, a side view, the steering mechanism 108 is assembled such that the steering mechanism cover 124 is sandwiched between tube body outer guide flange 126 and the steering cable drive pulley mounting chassis 130. The tube body inner guide flange 128 to which the outer guide flange 126 is assembled is not visible in FIG. 7A. Steering cable 116a is visible, and behind steering cable 116a is steering cable 116b, which is not visible.

FIG. 7B shows a front view of the symmetrical steering mechanism 108, and FIG. 7C shows a section view along section lines A-A. In FIG. 7B, the steering mechanism cover 124 and outer flange 126 are marked. The steering cables 116a, 116b are not marked, but the cables are visible and represented in a common plane. As seen in other views of the endoscopic device 100, the steering cables 116a, 116 are configured to deflect the steerable tip portion 106 in a first direction or a second direction in the common plane.

In the section view of FIG. 7C, several features of the steering mechanism 108 are illustrated as assembled. In addition to the tube member outer guide flange 126 and the steering cable drive pulley mounting chassis 130, the internal tube member inner guide flange 128 is also called out. Steering cable 116a, which is arranged inside a flexible tubular member 104 (not called out in FIG. 7C), passes from outside of the steering mechanism 108 into the steering mechanism 108. Also illustrated in FIG. 7C is the inter-cooperation of the pinion gear 132 and ring gear 138.

In FIG. 7D, a front side view of the steering mechanism 108 is presented. In FIG. 7D, the symmetrical steering mechanism cover 124 is shown, but the tube body outer guide flange 126 is not shown. A section view of the symmetrical steering mechanism cover 124, cut along section line A-A, is presented in FIG. 7E. The ring gear 138 is shown in FIG. 7E.

FIG. 8 is an exploded view of a symmetrical steering mechanism embodiment 108 from a rear perspective. Steering cables 116a, 116b, which are arranged in a tube body 104 (not shown in FIG. 8), pass into the steering mechanism 108 via the outer and inner tube guide flanges 126, 128. The steering mechanism cover 124, which is sandwiched between the tube guide flanges 126, 128, includes a ring gear 138. A dashed-dotted line indicates the cooperative alignment of the ring gear 138 to a pinion gear 132 when the steering mechanism is assembled. Also upon assembly, the shaft of the pinion gear 132 is aligned and affixed in a steering cable drive pulley 140 (FIG. 9) mounted in a chassis 130. An optional encoder 152 is cooperatively coupled to the shaft of the pinion gear 132.

As illustrated in FIGS. 7A-7E and FIG. 8, the ring gear 138 is illustrated as forming a completely circular gear. Optionally, the ring gear may also be formed as a geared surface that is not circular. For example, in some embodiments, ring gear 138 is formed in a partial circle pattern of 180 degrees or less. Each end of the partially formed ring gear 138 may include a stop feature arranged to prevent rotation of the symmetrical steering mechanism housing 124 when the pinion gear 132 reaches one or the other formed stop features.

The embodiment illustrated in FIG. 8 may be used to describe how steering cables 116a, 116b are controlled to cause a right and a left deflection of a steerable tip portion 106. In the embodiment, a rightward deflection of the steerable tip portion 106 occurs when the cover 124 to the symmetrical steering mechanism 108 is rotated in a counterclockwise direction. In the embodiment, a leftward deflection of the steerable tip portion 106 occurs when the cover 124 to the symmetrical steering mechanism 108 is rotated in a clockwise direction.

When the cover 124 is rotated clockwise (from the perspective of a medical practitioner holding the handle body 102), the ring gear 138 will also rotate clockwise, and the pinion gear and shaft 132 will rotate (from a perspective above the gear) counterclockwise. The counterclockwise rotation of the pinion gear and shaft 132 will draw the “left-side” steering cable 116a inward, which will cause a deflection of the steerable tip portion 106 to the left. When the cover 124 is rotated counter-clockwise, the ring gear 138 rotates counterclockwise, and the pinion gear 132 rotates clockwise. The “right-side” steering cable 116b will be drawn inward thus causing a deflection of the steerable tip portion 106 to the right. In other configurations and gearing arrangement, a clockwise rotation of the cover 124 will cause a rightward deflection of the steerable tip 106, and a counter-clockwise rotation of the cover 124 will cause a leftward deflection of the steerable tip 106.

In FIG. 9, one embodiment of a steering cable drive pulley 140 means is shown. The pulley 140 is keyed for a desired assembly to the shaft of a correspondingly keyed shaft of pinion gear 132. The “D” type keys of the illustrated shaft and pulley is representative only and other arrangements are possible to secure the shaft of the pinion gear 132 to the drive pulley 140. When the steering mechanism is assembled, a ring gear 138 (FIG. 8) turns when the steering mechanism housing 124 (FIG. 8) is rotated by a medical practitioner. The turning ring gear 138 differentially engages and forces rotation of the pinion gear 132, which correspondingly forces rotation of the shaft of the pinion gear 132. The rotating shaft, which is fixedly coupled to the steering cable drive pulley 140, forces the rotation of the pulley 140.

The shaft of the pinion gear 132 in FIG. 9 is optionally configured with an encoder 152. The encoder 152 illustrated in FIG. 9 is a rotary encoder, but other types of encoders may also be arranged. The encoder 132 is arranged to convert the angular position or motion of the pinion gear shaft 132 to an analog or digital code. In one embodiment, the encoder is a quadrature encoder that provides both the relative position and the direction of motion of the shaft of the pinion gear 132. In some cases, the quadrature encoder produces analog signals, which may be sine and cosine signals. In other cases, the quadrature encoder produces quadrature square wave signals that differentially represent the position and motion of the encoded shaft. A set of quadrature square wave signals from the encoder 152 is illustrated in FIG. 9.

In the embodiment of FIG. 9, the steering cable drive pulley 140 is a notched pulley that includes binding points 142a, 142b. Steering cable 116a is attached to the drive pulley 140 at a first binding point 142a, and steering cable 116b is attached to the drive pulley 140 at a second binding point 142b. When the drive pulley 140 rotates, as a result of the rotation of the steering mechanism housing 124 (FIG. 8), steering cable 116a or steering cable 116b is pulled and wound around the pulley 140.

Due to the relative sizes of the ring gear in the steering mechanism housing 124 and the pinion gear 132, a known rotation of the housing 124 is sufficient to rotate the drive pulley 140 by about one quarter turn. In one embodiment, a 30 degree rotation of the steering mechanism housing 124 (FIG. 8) is sufficient to rotate the drive pulley 140 by 90 degrees. In the embodiment, when the drive pulley 140 rotates 90 degrees, the steerable tip portion 106 of the tube body 104 is deflected 90 degrees. Other arrangements are also possible.

FIG. 10 shows another embodiment of a steering cable drive pulley 140 means. In the embodiment, instead of binding points affixed to the drive pulley 140 as shown in FIG. 9, a single steering cable 116 includes a binding bead 144a for a first portion 116a of the steering cable 116. The steering cable 116 also includes a second binding bead 144b attached to a second portion 116b of the steering cable 116. The binding beads 144a, 144b may be advantageously crimped or otherwise affixed to the steering cable 116 at appropriate locations.

The embodiment of FIG. 10 is one embodiment of an endoscopic device 100 that permits the flexible tubular member 104 to be separately manufactured and distributed from the disposable control handle shell 102. In such an arrangement, it is possible that a sterile tube body 104 of any length and composition is combined by a medical practitioner or other person to a sterile handle body 102. In such a case, the steering cable 116 having first and second binding beads 144a, 144b is passed into the handle body 102 through the flexible tubular member outer guide flange 126 (FIG. 8), through the flexible tubular member inner guide flange/steering cable guide (FIG. 8), and looped around the steering cable drive pulley 140. In such an embodiment, structures in the steering mechanism are arranged to particularly guide the steering cable 116 to the drive pulley 140, and the same or other structures are arranged to enable proper tension on the cable 116.

FIG. 11 shows an embodiment of a steering cable rack and pinion drive means 146. The rack and pinion system of FIG. 11 is an alternative to the drive pulley system of FIG. 10. A first steering cable 116a is arranged to couple to a first rack 148a, and a second steering cable 116b is arranged to couple to a second rack 148b. Between the first and second racks, a steering cable drive pinion gear 150 is attached to the shaft of the pinion gear 132. As shown in other figures, the pinion gear 132 is assembled in the steering mechanism 108 to cooperate with a ring gear 138 arranged in the steering mechanism cover 124. When the cover 124 is rotated, the ring gear also rotates, which forces the pinion gear and shaft to also rotate. As illustrated in FIG. 11, when the shaft of pinion gear 132 rotates, the steering cable drive pinion gear 150 also will rotate and cause the first rack 148a and the second rack 148b to move in opposite directions. The rearward motion of one rack pulls the associated steering cable and thus deflects the steerable tip portion 106 (FIGS. 5A-5C) in a first direction in a first plane. When the cover 124 is rotated in the opposite direction, the other rack will move rearward, thus pulling the associated steering cable and deflecting the steerable tip portion 106 in a second direction in the first plane.

FIGS. 12A and 12B each illustrate three views related to deflection of the steerable tip portion 106 as directed by operation of the rack and pinion drive means 146 of FIG. 11. In a first illustration of FIG. 12A, the rack and pinion drive means 146 is shown in a perspective view. The pinion gear 132 has been rotated in a clockwise direction as indicated. A first rack 148a has correspondingly been driven forward by the steering cable drive pinion gear 150. Oppositely, the second rack 148b has been driven rearward by the steering cable drive pinion gear 150, which has also pulled cable 116b. In the second illustration of FIG. 12A, the pinion drive means 146 is shown in a view from above. The offset racks 148a, 148b account for a difference ΔL between the relative positions of the first steering cable 116a and the second steering cable 116b. The different relative positions of the steering cables 116a, 116b cause the deflection of the steerable tip portion 106 in a first direction in a first plane.

Turning to FIG. 12B, the pinion gear 132 has been rotated in a counterclockwise direction. In contrast to each of the illustrations of FIG. 12A, the racks 148a, 148b and steering cables 116a, 116b have moved into an opposition position. Accordingly, the steerable tip portion 106 of FIG. 12B is also deflected in a direction opposite to the first direction in the first plane of FIG. 12. That is, in FIG. 12B, the steerable tip portion 106 is deflected in a second direction, opposite the first direction and in the same first plane.

FIG. 13 is an overlay section view of the handle portion 102 of a partially disposable endoscopic device 100. The figure illustrates a control module embodiment 112 entering an exemplary recess formed in the disposable control handle shell 102. The surfaces of the control handle shell 102 are shown in dashed lines to better understand the illustration. In FIG. 13, hinged end cap 114 has been opened, and the control module 112 has been partially inserted into a recess in the control handle 102. A reference arrow shows the direction of insertion.

When the control module 112 is fully inserted into the recess in the control handle 102, the control module 112 will cooperatively mate with the steering cable drive pulley mounting chassis 130 enclosed and retained within the handle shell. Certain non-limiting structural features are shown on the steering cable drive pulley mounting chassis 130. Different structural features could also be used. FIG. 13 illustrates by way of an asterisk an electrical coupling that is achieved between electric mechanical structures on a first face of the control module 112 and configuration of electrical contacts on the mounting chassis 130.

FIG. 14 is a perspective view of an exemplary control module 112. A top shell 154 is cooperatively coupled to a bottom shell 156. A closure seam 158 is illustrated in the control module 112. The shapes of the top and bottom shell structures 154, 156 are non-limiting, and other shapes are possible. The seam 158 may be formed by an ultrasonic weld, glue, a snap fit, screws, or some other fastening means.

Formed in the surface of the control module 112 are two exemplary mechanical registration features 160. In FIG. 14, the registration features 160 are illustrated as being molded in to the top shell structure 154 of the control module 112. In other embodiments, the registration features may have different shapes and in addition or alternatively different positions on the control module 112. The registration features 160 are formed for cooperative coupling with corresponding features in the recess of the control handle 102.

An input/output feature 162 is shown on the control module 112. In the exemplary embodiment of FIG. 14, the input/output feature is a power button 162. In some cases, the power button 162 is an electromechanical switch. In other cases, the power button 162 is an electrical contact below a flexible membrane. The input/output feature 162 may include tactile feedback, a visual output indicator, or some other feature. On a front surface of the control module 112, a configuration of electric contacts 164 are shown. In some cases, the contacts 164 are male pins (e.g., spring-loaded “pogo” pins). In other cases, the contacts 164 are female sockets. In still other cases, the contacts 164 are some other type of electrical contacts. When the control module 112 is assembled in the recess of the control handle 112, the contacts 164 electrically couple to corresponding electrical contacts in the control handle 102.

The control module 112 of FIG. 14 may be sealed such that the module 112 may be more readily sterilized, disinfected, sanitized, or otherwise medically cleaned. In some cases, the control module 112 may be wiped with a treated cloth or even bathed in a high-level disinfectant.

FIG. 15 is an exploded view of the control module 112 of FIG. 14. At a first surface (i.e., a front surface in FIG. 15), the set of electrical contacts 164 may include o-rings or other types of sealing means 168. The electrical contacts 164 are mounted on a shaped circuit board or other substrate 170, and the contacts 164 are assembled to pass through a face plate 166. In the embodiment of FIG. 15, when the control module 112 is assembled, the face plate 166 forms an external surface of the control module 112. The assembly of the face plate 166, shaped substrate 170, and control module shell housings 154, 156 may include certain structures such as gaskets or other means to seal the control module 112.

A substrate 172 (e.g., a circuit board or an integrated circuit) includes operative electronic circuitry. Detail A in FIG. 15 lists several components of the operative electronic circuitry that may reside on the substrate 172. The components may include a power supply/power regulator device, one or more microprocessors, memory, a user interface, one or more transceivers, clock circuits, input/output structures, one or more communications ports, an optional vibrator device, one or more audio devices, and an optional encryption module. In some cases, one or more of the components are formed on a single integrated circuit. In other cases the components may be discretely placed on a circuit board.

The power supply/power regulator circuits may include charge and/or discharge circuitry for a battery 174, which is also assembled in the control module 112. The power supply/power regulator may also include other circuits to provide or distribute power to modules on the substrate or modules controlled by a microprocessor.

A microprocessor mounted or formed on the substrate 172 may include a single central processing unit (CPU) or a plurality of CPU's. Each CPU may have one or more cores that execute software instructions. Various clocking circuits and devices such as system clocks may be used to operate a CPU, memory, and other circuits. Real-time clock devices can be used, for example, to set alarms or trigger events, time stamp and date stamp certain data, control battery recharging circuits, and for other purposes.

The memory on the substrate 172 may include volatile memory (e.g., RAM) and nonvolatile memory (e.g., ROM). Within the memory, one or more software programs may reside including, for example, an operating system, a presentation program, a user interface program, and one or more communications programs.

The memory on the substrate 172 may store instructions and data retrieved and acted on by the microprocessor. An operating system or another type of control loop may include application and driver programs that permit additional software to control the operations of the partially disposable endoscopic device 100. For example, particular programs can be used to accept user input and to provide system output through a variety of input/output structures.

In such cases, the memory is a non-transitory computer readable medium (CRM). The CRM is configured to store computing instructions executable by a CPU. The computing instructions may be stored individually or as groups of instructions in files. The files may include functions, services, libraries, and the like. The files may include one or more computer programs or may be part of a larger computer program. Alternatively or in addition, each file may include data or other computational support material useful to carry out the computing functions of a partially disposable endoscopic device 100.

Buttons, keypads, computer mice, memory cards, serial ports, bio-sensor readers, touch screens, and the like may individually or in cooperation be useful to an operator of the endoscopic device 100. The devices may, for example, input control information into the device 100. Displays, printers, memory cards, LED indicators, audio devices (e.g., speakers, piezo device, etc.), vibrators, and the like are all useful to present output information to the operator of the endoscopic device 100. In some cases, the input and output devices are directly coupled to the substrate 172 and electronically coupled to the CPU or other operative circuitry. In other cases, the input and output devices pass information via one or more communication ports (e.g., RS-232, RS-485, infrared, USB, etc.)

One or more transceivers may be arranged on the substrate 172. The transceivers provide unidirectional or bidirectional communications with the electronic circuits of the partially disposable endoscopic device 100. The transceivers may be arranged to communicate over short distances (e.g., personal area networks, direct device-to-device communications) or long distances (commercial cellular services such as GSM, CDMA, etc.). In some cases, a Bluetooth transceiver is provided. In some cases, an IEEE 802.11 WiFi transceiver is provided. In some cases, a cellular transceiver chipset is provided. Other wireless and wired communication transceivers may also be provided. The control module 112 of FIG. 15 includes a shaped antenna 176 for use with one or more transceivers. In the embodiment of FIG. 8, the shaped antenna is a flexible strip-type antenna, but in other embodiments, different types of antennas may be used. The flexible nature of antenna 176 allows for shape conformation to a mating surface, in this case the interior surface of the control module shell 154.

The one or more transceivers of the endoscopic device 100 can be configured to communicate control information, multimedia (i.e., audio/video) information, a patient's personal healthcare information, or other information. In cases where a patient's personal healthcare information is communicated, an encryption module may obfuscate the data prior to communication.

FIG. 16 illustrates a top view and a section view of the control module 112 of FIG. 14. The illustrations of the control module 112 in FIG. 16 are exemplary, and other arrangements are possible. In the top view, the mechanical registration features 160, the input/output feature 162, and the top shell 154 are referenced. A section line A-A is provided in the top view to indicate where the section view is cut away.

In the section view A-A, two contacts of the configuration of electric contacts 164 are shown. In FIG. 16, the contacts are pogo pins protruding from the control module 112. When the control module 112 is assembled in the recess of a control handle 102, the configuration of pogo pins 164 makes contact with a corresponding set of contacts in the control handle 102.

To help facilitate the electrical coupling, section view A-A illustrates a contact surface of the mechanical registration features 178. The registration features 178 are also shown as identified in Detail B. In the embodiment, the registration feature is arranged with angle 8. When the control module 112 is assembled in the recess of a control handle 102, the registration features 178 cooperate with corresponding features in the control handle 102 to align and properly seat the control module 112 for reliable electrical coupling.

The battery 174 and substrate 172 are illustrated in the Section A-A view of FIG. 16. Other features are also illustrated but not necessarily referenced. At the back of the control module 112, a rear contact surface 180 is referenced. In some embodiments, the rear contact surface 180 is arranged to present a configuration of electrical contacts and a supporting structure around the electrical contacts. In such embodiments, the control module 112 may be placed in a battery recharging unit, and the rear contact surface 180 provides features to align and support control module in the recharging unit.

FIG. 17 presents front and back views of the control module 112 embodiment of FIG. 14. In the front view, the configuration of electrical contacts 164 is arranged as a 2×7 array of pogo pins. Other arrangements are possible. In some cases, all 14 pins of the array passed electrical signals between the control module 112 and components associated with the endoscopic device 100. In other cases, one or more of the pins of the array 164 are used to help align the electrical contacts of the array 164.

In one embodiment, two pins of the array 164 are arranged for each of several features of the endoscopic device 100. For example, two pins may be configured to supply power signals to the endoscopic device 100. Power may be used within the control handle 102 and in the alternative or in addition, power may be distributed through the flexible tubular member 104 to the steerable tip portion 106.

Two pins of the array 164 may be used to provide electronic control of a liquid (e.g., water) source. The two pins may, for example, control a solenoid, an electronic valve, or some other mechanism that turns on and off a water source in which the water is passed through a lumen in the flexible tubular member 104 to the steerable tip portion 106. Two other pins may control a similar mechanism that turns on and off a gas (e.g., air) source that provides gas to the steerable tip portion 106. Yet two more pins may control a similar mechanism that turns on and off a source of suction. When the suction source is turned on, an aperture at the steerable tip portion 106 draws liquids, gases, and other material from the space around the steerable tip portion 106. Two additional pins of the array 164 may turn on and off a light source provided at the steerable tip portion 106. Two or more pins may control and pass the data to and from an imaging device at the steerable tip portion 106. For example, the steerable tip portion 106 may include an electronic image sensor configured to capture still or moving images. In such cases, individual images or full motion video signals can be captured at the steerable tip portion 106 of the partially disposable endoscopic device 100 and communicated to a presentation device. Additional pins of the array 106 may be spare pins or pins configured for some other purpose.

As illustrated in the back view of the control module 112 (FIG. 17), a four-pin array 182 is arranged in the rear contact surface 180. In one embodiment, the four-pin array 182 conforms to a USB port. When the control module is placed in a compatible device, power may be passed through to pins of the array 182. Power may be used to charge the battery 174 and to operate the electronic components on the substrate 172. Two other pins of the array 182 may be used to communicate information to and from the communication module 112.

FIG. 18 illustrates a partially disposable endoscopic device 100 embodiment being used in a medical procedure. With respect to FIG. 18, a non-limiting exemplary use of the partially disposable endoscopic device 100 will be described. In the medical procedure, a patient has exhibited symptoms of abdominal distress. A medical practitioner will use a partially disposable endoscopic device 100 to safely, effectively, and efficiently diagnose the condition inside the patient's stomach.

Prior to beginning the medical procedure, the medical practitioner properly attires himself and prepares clean hands for the procedure. The medical practitioner removes a disposable portion of the endoscopic device 100 from sterile packaging.

In some cases, the disposable portion of the endoscopic device 100 includes a preassembled a control handle 102 and tubular body member 104. In other cases, the control handle 102 is separately packaged. A medical practitioner may choose a tubular body member 104 or preassembled endoscopic device based on certain features arranged within the tubular body 104. Some features that the medical practitioner may consider are the length of the tubular member, the diameter of the tubular member, the type of imaging device arranged in the flexible tip portion 106, the number or size of lumens, the type of tools included in the flexible tip portion 106, and many other things.

After releasing the disposable portion from its sterile packaging, the medical practitioner may remove and dispose of his gloves and don a new pair of sterile gloves.

If the disposable control handle 102 and the tubular body 104 are not preassembled, the medical practitioner will assemble the two components together to form the disposable portion of the partially disposable endoscopic device 100. When the disposable portions are assembled, the medical practitioner will expose a recess in the disposable control handle 102. The recess may be exposed, for example, by releasing a catch or interlock mechanism on the upper or lower housing 120, 122 of the control handle 102. Releasing the catch may open an access panel (e.g., hinged end cap 114), thus exposing the recess. Subsequently, the medical practitioner or another person may advance a medically clean, reusable control module 112 into the recess. The amount of pressure necessary to advance the control module 112 into the recess may be a function of springs or particular registration features molded or otherwise formed in the structures of the control handle 102 and the control module 112.

Upon closing the access panel, operation of the endoscopic device 100 may be verified after pressing a user interface input/output 136 in the control handle. Applying pressure to one or more of the user interface I/O 136 features will engage a power button on the control module 112, thereby turning on the endoscopic device 100. In some cases, lights, sounds, vibrations, or some other feedback will inform the medical practitioner that the device is operating. The medical practitioner may also operate other user interface I/O 136 features to test the various functions of the endoscopic device 100.

In some embodiments, the endoscopic device 100 includes a wireless 802.11 WiFi transceiver. When the endoscopic device 100 is powered, the transceiver provides signaling data to external computing devices 188. An external computing device 188 in the endoscopic device 100 may form a communicative coupling as a matched pair of devices. Information may be passed between the computing device 188 and the endoscopic device 100. A presentation device 190 attached to the computing device 188 informs the medical practitioner that a communication link has been established. Subsequently, information associated with the endoscopic device 100 can quickly and efficiently be passed to the computing device 188. The computing device 188 may be a computer and video display mounted or placed in a surgery suite, a portable tablet computing device, a smart phone, a medical device such as a fluoroscope or ultrasound machine, or any other type of computing device configured to receive wireless communication.

Prior to use in the medical procedure, the medical practitioner may test other features. For example, if the endoscopic device 100 includes a light output, the practitioner may manipulate user interface I/O structures 136 on the control handle 102 to turn on and off the light. If the endoscopic device 100 includes an image sensor, the practitioner may manipulate user interface I/O structures 136 on the control handle 102 to adjust brightness, contrast, color, or other features.

If the endoscopic device 100 includes an image sensor device, the medical practitioner may test the image sensor by turning it on via the user interface I/O 136. The image sensor device may be configured for image capture or video acquisition. Accordingly, the medical practitioner may aim the image sensor embedded in the steerable tip portion 106 toward any recognizable object. The medical practitioner may expect to see a representation of the object on a presentation device 190 coupled to the computing device 188. Still images or full or partial motion video may be presented. Images or video may be recorded and played back. The computing device may include other features to direct or control the information captured by the image sensor.

The medical practitioner may further test the endoscopic device 100. Water or other liquid sources may be coupled to the endoscopic device 100 such that the liquid is passed through a lumen in the tubular body 104. The function may be controllable via the user interface I/O 136. Similarly, the medical practitioner may test an air source or other gas source and a suction source. If the endoscopic device 100 includes other tools that perform functions in the steerable tip portion 106, the medical practitioner may test them. Alternatively, or in addition, the medical practitioner may pass other tools through a lumen in the tubular body 104, thereby testing the integrity of the lumen and the ability to control the tools.

The medical practitioner may test the single-handed, either-handed operation of the symmetrical steering mechanism 108 by rotating the symmetrical steering mechanism cover 124. To perform this test, the medical practitioner may place the control handle 102 in the palm of either his left hand or his right hand. Using his thumb and/or index finger of the hand that is holding the control handle 102, the practitioner may rotate the steering mechanism cover 124. Upon rotating the steering mechanism cover 124, the medical practitioner will see the flexible tip portion 106 deflect one direction (e.g., to the right) or an opposite direction (e.g., left). The steerable tip portion 106 will move in one direction or the other direction in a common plane. In some embodiments, the steerable tip portion 106 is permitted to move 270° in the common plane. In other embodiments, the steerable tip portion 106 may only move 180° in common plane.

The proper operation of the steering mechanism 108 can be confirmed by the medical practitioner in many ways. For example, the medical practitioner can visually inspect the motion of the flexible tip 106 when the steering mechanism cover 124 is rotated. The medical practitioner can observe the presentation device 190 when the image sensor 198 is enabled. As the flexible tip moves, the image or images represented on the presentation device 190 will change accordingly. The medical practitioner can also observe the data passing from an encoder 152 if such a device is included. Data from a rotary encoder 152 may be processed and presented as a degree of rotation and direction on the presentation device 190.

The medical practitioner may perform further testing of the symmetrical steering mechanism 108. The practitioner may switch hands that are holding the control handle 102. The practitioner may use one hand to hold the control handle 102 while using his other hand to turn the steering mechanism cover 124. During the testing, the medical practitioner may also be using the image sensing features, lights, water nozzles, air nozzles, suction sources, and other features of the endoscopic device 100 to facilitate testing.

In some cases, a medical practitioner prefers to hold the control handle 102 in his dominant hand. In this arrangement, the medical practitioner can easily control the symmetrical steering function and other user I/O functions of the control handle 102. Concurrently, the medical practitioner will use his subordinate hand to control the flexible tubular member 104 as it is advanced into a patient's body. In such an arrangement, the medical practitioner can rotate the torque stable tubular body 104 using his subordinate hand as he passes the tube body 104 into the patient. At the same time, the medical practitioner can steer the flexible tip 106 in a common plane using his dominant hand. As the medical practitioner's two hands cooperate to steer and rotate the flexible tip 106, the flexible tip 106 can be pointed in any direction in three-dimensional space.

After adequately testing the functions of the endoscopic device 100 to his satisfaction, the medical practitioner can begin the medical procedure. The patient has been properly prepared for the procedure.

The medical practitioner begins the procedure by lubricating the flexible tubular member 104. In the procedure, the tubular member will be passed down the patient's throat. As the medical practitioner advances the flexible tip 106 into the patient's mouth, he can confirm the direction and location of the flexible tip by viewing the presentation device 190. The practitioner may turn on a light 202 in the functional module 110 of the flexible tip 106 to illuminate the path in front of the tip 106. The medical practitioner may also choose to record the streaming video that is being presented.

The medical practitioner can steer the flexible tip 106 by cooperatively rotating the symmetrical steering mechanism cover 124 and by rotating the entire control handle 102. In some cases, the medical practitioner will desirably change which hand is holding the control handle 102 as he rotates the device. The symmetrical nature of the endoscopic device 100 allows the practitioner to switch hands as seldom or frequently as he chooses.

The flexible tube 104 is further steered and advanced past the patient's throat, through the esophagus, and into the patient's stomach. The medical practitioner may enable an air nozzle 196 to inflate the stomach 18. He may also apply water from the water nozzle 194 to clean the light source 202 and a lens or other surface in front of the image sensor 198.

In the medical procedure illustrated in FIG. 18, the medical practitioner has identified an area of interest 184 in the stomach 18 of the patient. The medical practitioner can see the represented area of interest 186 on the presentation device 190. The control module 112 or the computing device 188 may include image recognition software. In such a case, the medical practitioner may receive additional computational assistance to determine the type or condition of particular tissue being captured by the image sensor 198.

In some cases, the medical practitioner may pass a tool through a tool lumen 192 in the endoscopic device 100. The tool may be, for example, a forceps tool to sample or excise the area of interest 184. Alternatively, or in addition, the practitioner may apply a different therapy to the area of interest.

After completion of the medical procedure, the medical practitioner will withdraw the flexible tube 104 from the patient.

When the flexible tube 104 has been withdrawn, the medical practitioner will power down the endoscopic device 100. The practitioner will then find a clean receptacle. Upon locating a clean receptacle, the practitioner will again open the access port (e.g., hinged end cap 114) of the endoscopic device 100. When the access port is opened, the control module 112 is permitted to drop into the receptacle. The control module, which had previously been enveloped in the hermetically sealed recess of the control handle 102, has not been contaminated with any biological agents of the patient, medical practitioner, or anyone else. Out of an abundance of caution, the control module 112 will nevertheless be sealed in the receptacle to later be medically cleaned. The control module 112 is reusable and is not disposed of.

After the control module 112 has been released from the control handle 102, the medical practitioner will properly dispose of the control handle 102 and flexible tube 104. The assembly formed by the control handle 102 and the flexible tube 104 was sterile when the medical procedure began, and the assembly is disposed of after the medical procedure. The assembly will not be used on another patient. Accordingly, a patient can be given assurance that no infectious agents or other contaminants between this patient and another patient as a result of the medical procedure.

As described herein, for simplicity, a medical practitioner is in some case described in the context of the male gender. For example, the terms “his hand,” “his left thumb,” and the like are used. It is understood that a medical practitioner can be of any gender, and the terms “he,” “his,” and the like as used herein are to be interpreted broadly inclusive of all known gender definitions.

As described herein, the terms “rigid” and “semi-rigid” may be interchanged. Accordingly, “rigid” device is not necessarily completely unbendable. Instead, a rigid device or a rigid portion of a device has a desired degree of stiffness. That is, a device that is “rigid” or “semi-rigid” is a device that resists deformation to a desired degree. The desired degree of rigidity may be measured, for example, in units such as foot pounds per inch or some other units. One device may be more rigid than another device. The increased (or decreased) rigidity may be caused by the devices being formed from different materials, from materials having different physical or chemical properties, or for some other reason. Correspondingly, the terms “flexible,” “flexibility,” and the like impart a desired degree of flexibility to the device which the term modifies.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

As used herein and in the appended claims, the singular forms “a”, “an”, and “the” include plural referents unless the context clearly dictates otherwise. It should also be noted that the term “or” is generally employed in its sense including “and/or” unless the content clearly dictates otherwise.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

The headings and Abstract of the Disclosure provided herein are for convenience only and do not interpret the scope or meaning of the embodiments.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

1. An endoscopic device, comprising:
a disposable control handle having a sealable recess therein;
a disposable flexible tubular member coupled to the control handle; and
a non-disposable electronics control module configured for removable placement within the sealable recess.

2. The endoscopic device of claim 1 wherein the disposable control handle and the disposable flexible tubular member are sterilized and packaged for use on a single patient.

3. The endoscopic device of claim 1 wherein the non-disposable electronics control module is configured to be disinfected and re-used on two or more patients.

4. The endoscopic device of claim 1 wherein the disposable control handle includes a hinged access panel, the hinged access panel providing an entryway to the sealable recess.

5. The endoscopic device of claim 1 wherein the disposable control handle and the non-disposable electronics control module include cooperative registration features configured to align a first plurality of electrical contacts arranged on the non-disposable electronics control module with a second plurality of corresponding electrical contacts arranged in the sealable recess of the disposable control handle.

6. The endoscopic device of claim 1 wherein the disposable flexible tubular member, comprises:
a first one or more wires embedded in the disposable flexible tubular member, the first one or more wires electrically coupled to a light source integrated in a distal end of the disposable flexible tubular member, the first one or more wires arranged to pass at least one first signal from the non-disposable electronics control module to the light source; and
a second one or more wires embedded in the disposable flexible tubular member, the second one or more wires electrically coupled to an image sensor integrated in the distal end of the disposable flexible tubular member, the second one or more wires arranged to pass at least one second signal from the non-disposable electronics control module to the image sensor.

7. The endoscopic device of claim 6 wherein the non-disposable electronics control module, comprises:
a wireless transceiver configured to communicate image data captured by the image sensor to a remote computing device.

8. The endoscopic device of claim 7 wherein the remote computing device is at least one of a computer, a portable tablet computing device, a smart phone, or a medical computing device configured to receive wireless communication.

9. The endoscopic device of claim 6 wherein the non-disposable electronics control module, comprises:
a multi-pin port configured to pass power into the non-disposable electronics control module and further configured to communicate data between the non-disposable electronics control module and a remote computing device.

10. The endoscopic device of claim 1 wherein the disposable flexible tubular member, comprises:
a first lumen arranged to pass a liquid through the disposable flexible tubular member and out of a distal end of the disposable flexible tubular member, the passage of the liquid controllable via at least one first signal from the non-disposable electronics control module;
a second lumen arranged to pass a gas through the disposable flexible tubular member and out of the distal end of the disposable flexible tubular member, the passage of the gas controllable via at least one second signal from the non-disposable electronics control module; and
a third lumen arranged to draw material via suction through the disposable flexible tubular member and from the distal end of the disposable flexible tubular member, the non-disposable electronics control module configured to generate at least one third signal to enable the suction.

11. A method to use a partially disposable endoscopic device, comprising:
arranging a sterile disposable control handle having a sealable recess therein in the vicinity of a patient's body, the sterile disposable control handle having a sterile disposable flexible tubular member coupled thereto;
opening the sealable recess in the disposable control handle;
introducing a non-disposable electronics control module into the sealable recess;
performing a medical procedure that includes passing the sterile disposable flexible tubular member into the patient's body and controlling at least one feature of the disposable flexible tubular member with the disposable control handle;
re-opening the sealable recess in the disposable control handle;
removing the non-disposable electronics control module from sealable recess; and
disposing of the sterile disposable control handle and the sterile disposable flexible tubular member.

12. The method to use the partially disposable endoscopic device of claim 11, comprising:
disinfecting the non-disposable electronics control module; and
packaging the disinfected non-disposable electronics control module for use in another medical procedure.

13. The method to use the partially disposable endoscopic device of claim 11 wherein medical procedure includes passing at least a portion of the disposable flexible tubular member through the esophagus of the patient's body.

14. The method to use the partially disposable endoscopic device of claim 11, comprising:
controlling, during the medical procedure, an image sensor integrated in the distal end of the disposable flexible tubular member via at least one signal passed from the non-disposable electronics control module; and
wirelessly communicating at least one image captured by the image sensor from the partially disposable endoscopic device to a remote computing device.

15. The method to use the partially disposable endoscopic device of claim 11, comprising:
exposing the partially disposable endoscopic device as a wirelessly discoverable device;
communicatively coupling the partially disposable endoscopic device to a wireless computing device; and
wirelessly communicating image data captured by the image sensor from the partially disposable endoscopic device to the communicatively coupled wireless computing device.

16. An endoscopic device, comprising:
a control handle configured for single-handed, either-handed operation; and
a steering mechanism cooperatively assembled with the control handle, the steering mechanism arranged to steer at least one portion of a flexible tubular member attachable to the steering mechanism.

17. The endoscopic device of claim 16, comprising:
a rotatable housing integrated with the steering mechanism, the rotatable housing configured for rotation by at least one of five digits of a right hand and by at least one of five digits of a left hand.

18. The endoscopic device of claim 17 wherein the steering mechanism comprises:
a system having at least two geared structures and at least two steering cables, the at least two geared structures and at least two steering cables arranged to deflect a distal end of an attached flexible tubular member at least 180 degrees in a common plane.

19. The endoscopic device of claim 18 wherein the steering mechanism is arranged to deflect the distal end of an attached flexible tubular member at least 100 degrees in a positive direction of the common plane when the rotatable housing is rotated in a first direction, the steering mechanism further arranged to deflect the distal end of an attached flexible tubular member at least 100 degrees in a negative direction of the common plane when the rotatable housing is rotated in a second direction.

20. The endoscopic device of claim 16 wherein the steering mechanism is arranged to steer the at least one portion of an attached flexible tubular member during a first time when the control handle is in one of a left hand and a right hand, the steering mechanism further arranged to steer the at least one portion of the attached flexible tubular member during a second time when the control handle is in the other of the left hand and the right hand.
